(19) United States
(12) Patent Application Publication
(10) Pub. No.: US 2025/0257241 A1
(43) Pub. Date: Aug. 14, 2025

(54) Formulations Containing Biologically Prepared Fibers

(71) Applicant: Battelle Memorial Institute, Columbus, OH (US)

(72) Inventors: Fadime KARA MURDOCH, Upper Arlington, OH (US); Heather LUCKARIFT, Port St Joe, FL (US); Cheryl IMMETHUN, Columbus, OH (US); Jennifer BROOKS, Hilliard, OH (US); Bradley HEATER, Dublin, OH (US); Rachel KREBS, Hilliard, OH (US)

(73) Assignee: BATTELLE MEMORIAL INSTITUTE, Columbus, OH (US)

(21) Appl. No.: 19/049,896

(22) Filed: Feb. 10, 2025

Related U.S. Application Data
(60) Provisional application No. 63/551,935, filed on Feb. 9, 2024.

(51) Int. Cl. C09D 189/00; C04B 41/00; C04B 41/45; C04B 41/48; C04B 41/63; C09D 7/20; C09D 7/40; C09D 7/63; C09D 101/02; C12N 9/88

(52) U.S. Cl. CPC C09D 189/00; C04B 41/009; C04B 41/4596; C04B 41/4803; C04B 41/4807; C04B 41/63; C09D 7/20; C09D 7/63; C09D 7/70; C09D 101/02; C12N 9/88; C12Y 402/01001

(57) ABSTRACT

Disclosed herein are formulations comprising biologically prepared fibers and methods of preparing said formulations. These formulations can be utilized for repair of structures comprised of a variety of materials, including cementitious material, natural rock, mortar, and concrete. The formulation includes biologically prepared fibers, and a solvent or carrier solution. The formulation may additionally and optionally incorporate at least one enzyme. Once the formulation containing the biologically prepared fibers is applied to a void or crack within the structure, the fibers form a three-dimensional fibrous scaffold configuration in the void of the structure.

### CROSS-REFERENCE TO RELATED APPLICATIONS

This Application claims priority to and benefit of U.S. Provisional Patent Application No. 63/551,935, titled “FORMULATIONS CONTAINING BIOLOGICALLY PREPARED FIBERS”, filed on Feb. 9, 2024, the contents of which are incorporated by reference herein in its entirety.

### STATEMENT REGARDING FEDERALLY SPONSORED RESEARCH AND DEVELOPMENT

This invention was made with Government support under Contract CNTR000000000000866 awarded by the Defense Advanced Research Projects Agency (DARPA). The U.S. government has certain rights in the invention.

### FIELD OF INVENTION

The current application relates to formulations containing biologically derived fibers and methods of preparing said formulations. More specifically the invention pertains to biologically derived fibers used in adhesive or repair formulations for cementitious materials, natural rocks, mortars, concrete, and the like.

### BACKGROUND

Current repair methods for cementitious materials require surface application with limited penetration depth into cracks or voids. As such, current methods and compositions are limited in efficacy of repair. Biological approaches have been previously investigated. These approaches typically require the biological component to be incorporated into the cementitious material at time of manufacture and are not suitable for application to existing aged infrastructure.

There remains a need for biologically derived formulations that are easy to implement with aging infrastructure and can be applied directly to cracks or voids of cementitious materials or surfaces for repair thereof.

### SUMMARY

Disclosed herein are formulations comprising biologically prepared fibers and methods of preparing said formulations. These formulations can be utilized for repair of structures comprised of a variety of materials, including cementitious material, natural rock, mortar, concrete, with various aggregate and cement ratios, and various aggregate and cement formulations and so on.

In one embodiment, a formulation for repair of a structure is disclosed. The formulation includes: biologically prepared fibers, and a solvent or carrier solution. The formulation may additionally and optionally incorporate at least one enzyme. Once the formulation containing the biologically prepared fibers is applied to a void or crack within the structure, the fibers form a three-dimensional fibrous scaffold configuration in the void of the structure. As such, the fibers can be utilized as a delivery/transport and support means for nutrients within the void. For example, a carbonic anhydrase enzyme can be injected into the void which catalyzes mineral formation (calcite formation) and as such repairs the void structure.

In one embodiment, the biologically prepared fibers are from silk fibroin (SF) fibers. In another embodiment, the biologically prepared fibers are bacterial cellulose (BC) fibers. In a further embodiment, a combination of both SF and BC fibers in various ratios are used in the formulation.

In certain embodiments, at least one enzyme is added to the formulation comprising the biologically prepared fibers. The enzyme can be added prior the formulation being injected or applied to a crack or void to be repaired in a structure. Alternatively, a solution containing the enzyme can be applied or injected into the crack, void or surface to be repaired, after the solution containing the biological fibers has already been applied and formed the three-dimensional scaffold configuration. In one embodiment, the enzyme comprises carbonic anhydrase (CA).

Methods for repair of structures are also disclosed. In one embodiment, a method for repairing a structure comprises the steps of: applying a first solution comprising biologically prepared fibers to the structure and optionally, applying a second solution containing at least one enzyme to the structure, wherein the biologically prepared fibers comprise Silk Fibroin (SF), or Bacterial Cellulose (BC), or a combination thereof.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all subcombinations of the various embodiments and elements thereof are also specifically embraced by the present invention and are disclosed herein just as if each and every such subcombination was individually and explicitly disclosed herein.

### Selected Definitions and Nomenclature

As used herein the term “biologically prepared fibers” or “biological fibers” refers to fibers at the nano or micro-scale which are derived from biological living organisms, including silk worm and bacterial organisms. The term does not refer to plant derived fibers, such as cellulose fibers derived from plant materials.

The singular forms “a”, “an” and “the” are intended to include the plural forms as well, unless the context clearly indicates otherwise.

The term “and/or” or “and or” refers to and encompasses any and all possible combinations of one or more of the associated listed items.

The term “about,” when referring to a measurable value such as length, width, diameter, radius, or an amount of a compound, dose, time, temperature, and the like, is meant to encompass variations of 10%, 5%, 1%, 0.5%, or even 0.1% of the specified amount.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure

As used throughout this description, and in the claims, a list of items joined by the term “at least one of” or “one or more of” can mean any combination of the listed terms. For example, the phrase “at least one of X, Y or Z” can mean X; Y; Z; X and Y; X and Z; Y and Z; or X, Y and Z.

All patents, patent applications and publications referred to herein are incorporated by reference in their entirety.

### BRIEF DESCRIPTION OF THE DRAWINGS

FIG. 1 illustrates a prepared specimen and evaluation of crack repair lengthwise and cross-sectional widthwise.

FIG. 2 illustrates images gap fugitive microcosm treated with bacterial cellulose (BC) fibers. Images A and C, and B and D have 0.8 mm gap with BC filled microcosm. Images E and F have a 0.5 mm microcosm.

FIG. 3 shows a p-Nitrophenyl esterase assay of CA after entrapment within the silk fibers. Results of esterase activity are shown as a function of increasing enzyme amount.

FIG. 4 shows Cycle 1 of p-Nitrophenyl esterase assay of CA after entrapment within the silk fibers. Results of esterase activity are shown as a function of decreasing enzyme amount.

FIG. 5 shows Cycle 2 of p-Nitrophenyl esterase assay of CA after entrapment within the silk fibers.

FIG. 6 shows a p-Nitrophenyl esterase assay of CA after entrapment within the bacterial cellulose fibers, as a function of increased enzyme.

FIG. 7 shows a p-Nitrophenyl esterase assay of CA after entrapment within the bacterial cellulose fibers, as a function of decreasing enzyme amount.

FIG. 8 illustrates graph of crack filling % in airfield concrete samples.

FIG. 9 illustrates graph of crack filling rate % in mass concrete samples.

FIG. 10 illustrates graph of crack filling rate % in marine concrete samples.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative bases for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical application. Various combinations and modifications of the features consistent with the teachings of this disclosure, however, could be desired for particular applications or implementations.

In one embodiment, a formulation for repair of a structure is disclosed. The formulation includes: biologically prepared fibers, and a solvent or carrier solution. The formulation may additionally and optionally incorporate at least one enzyme. Once the formulation containing the biologically prepared fibers is applied to a void or crack within the structure, the fibers form a three-dimensional fibrous scaffold configuration in the void of the structure. As such, the fibers can be utilized as a delivery/transport and support means for nutrients within the void. For example, a carbonic anhydrase enzyme can be injected into the void which catalyzes mineral formation (calcite formation) and as such repairs the void structure.

In one embodiment, the biologically prepared fibers are from silk fibroin (SF) fibers. In another embodiment, the biologically prepared fibers are bacterial cellulose (BC) fibers. In a further embodiment, a combination of both SF and BC fibers in various ratios are used in the formulation.

In embodiments where the biologically prepared fibers comprise silk fibroin, the prepared fibers are present in the formulation at about 4-15% wt/vol %, or about 5-14% wt/vol, or 6-13% wt/vol or about 7-11% wt/vol, or about 8-10% wt/vol. In one embodiment, the biologically prepared silk fibroin fibers comprise water-insoluble silk II proteins in β-sheet formation. In certain embodiments, the SF fibers are combined with 20 mM calcium lactate, for purposes of effecting conversion of the protein from SF I to SF II, β-sheet formation and provide calcium source for calcite formation. Calcium lactate can be added to the SF containing formulation, prior to or post injection or application on a crack, void or surface to be repaired. Calcium lactate can be replaced with various calcium sources, including calcium chloride and calcium acetate. Hence, the β-sheet formation of the SF fibers can be actuated either prior to injection, or in a subsequent step, after injection into a crack or void has occurred (as part of a two-step process of first injecting the formulation, then injecting a second reactant in order to effect the β-sheet formation of the proteins, which ultimately leads to the scaffold formation configuration).

In embodiments where the biologically prepared fibers comprise bacterial cellulose fibers, the prepared fibers are present in the formulation at about 0.4-1.5% wt/wt, or about 0.5-1.4% wt/wt, or about 0.6-1.3% wt/wt. or about 0.5-1.2% wt/wt, or 0.6-1.1% wt/wt, or any range or value therebetween. In certain embodiments, the BC fibers are prepared and solubilized in a solvent. The solvent can comprise N-Methylmorpholine-N-Oxide monohydrate (NMMO.H₂O). In one embodiment, the BC fibers are solubilized at about 0.7% wt/wt in the NMMO.H₂O solvent. In further embodiments, the BC fibers are solubilized at about 1.2% wt/wt in the NMMO.H₂O solvent.

In certain embodiments, at least one enzyme is added to the formulation comprising the biologically prepared fibers. The enzyme can be added prior the formulation being injected or applied to a crack or void to be repaired in a structure. Alternatively, a solution containing the enzyme can be applied or injected into the crack, void or surface to be repaired, after the solution containing the biological fibers has already been applied and formed the three-dimensional scaffold configuration. In one embodiment, the enzyme comprises carbonic anhydrase (CA). The purpose of the enzyme is to catalyze mineral formation (i.e., calcite) within the void, crack or surface of application, so as to facilitate repair of the structure. In certain embodiments, commercial bovine carbonic anhydrase (bCA) and recombinant carbonic anhydrases (CA) are utilized, including examples such as CA derived from Sulfurihydrogenibium azorense (SazCA), CA from Thermosulfurimonas dismutans (TdCA), and a CA mutant from Bos taurus (bCAIIM4).

In embodiments, silk fibroin (SF) was prepared from Bombyx mori cocoons. To remove sericin coat proteins from silkworm cocoons (known as degumming), silkworm cocoons were boiled in 0.5% NaHCO₃, washed with Milli-Q water and the degummed cocoons were air-dried. They were dissolved in CaCl₂:C₂H₆O:H₂O (1:2:8 mole ratio) by heating up to 80° C. Then, the silk solution was dialyzed against deionized water (frequent changes of deionized water) at room temperature for at least three days to purify. After purification, the purified silk solution was aliquoted into clean containers for lyophilization. Primary drying (removal of unbound water) was conducted below the material's glass transition temperature, T_{g} (between −25.5 and −22° C.) to regenerate the silk (in water-soluble form). The final product is an amorphous solid and 100% water soluble. For transition of the protein into the β-sheet configuration (water insoluble form), the solution was briefly vortexed, or calcium lactate (20 mM) was added. The final silk fibroin concentration applied for crack filling is 6-10 wt/vol % SF. It was confirmed that SF creates a fibrous 3-dimensional structure within the crack/gap. The resulting SF repair imparts increased mechanical strength, fills the crack void, and adheres the cracked concrete surfaces together. The fibrous three-dimensional scaffold's configuration was validated through imaging using Field Emission Scanning Electron Microscopy.

In other embodiments, bacterial cellulose (BC) was produced by growing Gluconacetobacter xylimis on modified Hestrin-Shramm medium under static conditions at 28° C. The BC was harvested, purified, homogenized, and dried by lyophilization for 36-48 h or by oven-drying at 50° C. for 24-36 h. The dried BC fibers were dissolved in N-Methylmorpholine-N-Oxide monohydrate with addition of propyl gallate (0.2 wt % for cellulose, to avoid oxidation and degradation of BC while dissolving at high temperature) at 80° C. for 8-12 h with stirring and the solubilized BC was applied into natural cracks and/or manufactured gaps of concrete microcosms. It was confirmed that BC creates a fibrous 3-dimensional structure within the crack/gap. The resulting BC repair imparts increased mechanical strength, fills the crack void, and adheres the cracked concrete surfaces together. The structure of the fibrous 3-dimensional scaffold was confirmed by Field Emission Scanning Electron Microscopy imaging. The approach provides a simple, bio-based, room temperature “adhesive” for repair of concrete cracks in aged structures.

Methods for repair of structures are also disclosed. In one embodiment, a method for repairing a structure comprises the steps of:
applying a first solution comprising biologically prepared fibers to the structure;
optionally, applying a second solution containing at least one enzyme to the structure;
   wherein the biologically prepared fibers comprise Silk Fibroin (SF), or Bacterial Cellulose (BC), or a combination thereof.

In another embodiment, a method for repair of a structure is carried out in a single step. The method comprises:
preparing a solution comprising:
   biologically prepared fibers;
   a solvent; and
   at least one enzyme; and
applying the solution to the structure in a single application step;
   wherein the biologically prepared fibers comprise Silk Fibroin (SF), or Bacterial Cellulose (BC), or a combination thereof.

### EXAMPLES

### Example 1-Visualization of Vascularization of Biologically Prepared Fibers in Fugitive Microcosms

Good distribution and heterogeneous application of the vascular scaffold is essential for delivering the crack healing biologically prepared fibers and a protocol was developed to determine total percent fill and fill rate for the vascularization material, using a stereo microscope with image analysis software. Fugitive microcosms contain a single manufactured crack to assess crack-filling rate. Two halves of a saw-cut cylinder are pieced together using spacers of a pre-determined thicknesses (0.2, 0.5 and 0.8-mm). Once reassembled, masking tape is applied to the circumference to ensure the specimen remains together. Fugitive microcosms were used to monitor crack-filling rate to evaluate metrics for the application function. At various timepoints the samples will be sectioned and analyzed to visualize the location and integrity of the vascular structure across the width of the cylinder and as a visualization of integration depth. Continued (prolonged function) is recorded over time as a measurement of sustained healing through the crack.

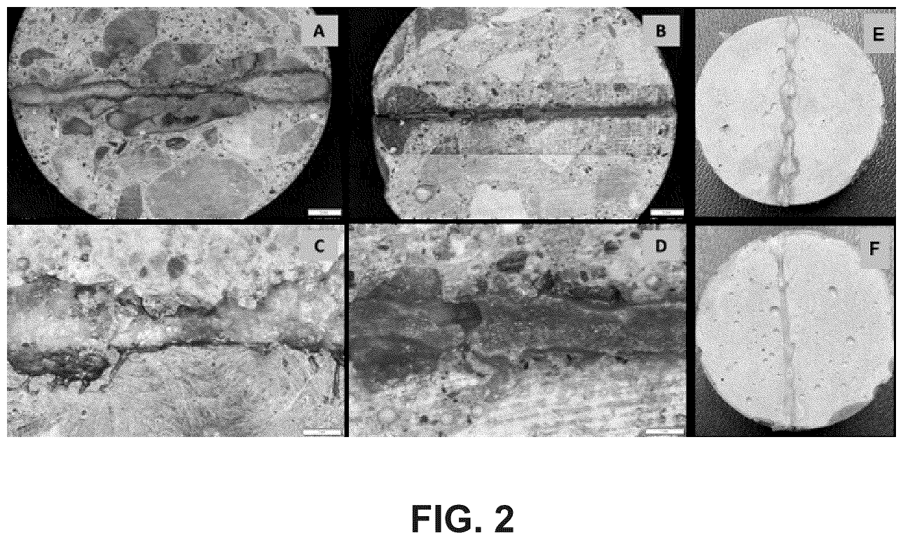

The simplest application is percolation from the upper most surface, although the rate will be dependent upon viscosity and transition rate (gelation and hardening). The volume added and the time to dry is recorded, and the process repeated until no more material will flow into the gap. Fill rate was measured by monitoring the time between adding vascularization material to the top of the microcosm to the time it emerges from the bottom. After adding the material, cross sections of ˜0.6″ were sliced with a wet saw. Images of the cross sections were then used to determine the percent fill, calculated using ImageJ's threshold function. The microcosms were imaged using an Olympus SZX12 Stereo Microscope with Olympus DP74 color fluorescence camera. Both bacterial cellulose (BC; dissolved in N-Methylmorpholine N-oxide monohydrate (NMMO.H₂O), 0.7 wt/wt %) and silk fibroin (SF; 5-6 wt/vol %) were tested using this protocol (results are shown in FIG. 2).

While SF can be seen in the grooves of the microcosm, it initially was not discernable from the concrete surface. Similarly, the BC solution could be seen in the gap at the bottom of the microcosm within seconds after application. The treated microcosms were allowed to dry overnight in the fume hood at room temperature. Next day, the microcosms were imaged for vascularization/filling (FIG. 2). FIG. 2 shows a 0.8 mm gap-fugitive microcosm treated with BC after overnight drying. Top images (A and C) and bottomiamges (B and D) are of the BC-filled fugitive microcosm. Top images (E) and bottom images (F) are of 0.5 mm fugitive microcosm.

For SF, green food coloring was the only dye that provided stable coloration and allowed the Silk I to Silk II confirmation change to occur. 6 wt/vol % SF dyed with green food coloring (with and without 20 mM calcium lactate) was applied to the 0.2 mm gap in fugitive microcosms. Both solutions flowed through the microcosms in seconds and were allowed to dry for 24 hours. The SF with the calcium lactate initiated the confirmation change immediately after application, which was visible on top of the microcosm. The two halves of the fugitive microcosms were pried apart to examine the coverage visually. The green SF without calcium lactate was seen across the inner surfaces of the microcosm, indicating good coverage and adhesion while the green SF with calcium lactate did not adhere since it had already converted to B-sheets. 5 wt/vol % SF was used for subsequent tests to counter this problem.

After some optimization studies related to fiber concentration and preparation of fibers, results confirmed good distribution of vascular biologically prepared fibers throughout the fugitive microcosms. Both SF and BC dried as a solid fill material that could not be separated by manual pressure to pry the two halves of the microcosm apart.

### Example 2-Addition of Enzyme to Cracked Microcosms

To use Carbonic Anhydrase (CA) for crack repair, the enzyme should be highly active, stable in the alkaline environment of concrete and exhibit stability over the month-to-year timescale. For CA enzymes, esterase activity was confirmed by measuring p-nitrophenyl acetate hydrolysis, which forms the yellow product p-nitrophenol. Although this assay is useful to determine the functionality of CA enzyme, it does not directly address calcite formation rate. Therefore, a simple assay was developed to compare calcite formation by adding calcium to the reaction and monitoring the enzymatic conversion of CO2 to bicarbonate that results in precipitation of calcium to form calcium carbonate/calcite. Crack repair rates of the different CAs were then determined directly in the concrete environment using the 0.2 mm cracked concrete microcosms. Aqueous solutions of the three different CAs and an enzyme-free control using 0.1 M CaCl2 in CAPS buffer pH 11 were added to the crack using a pipette. Most of the liquid absorbed into the concrete very quickly and spread out, so the crack would require multiple applications to completely fill.

The CA-catalyzed formation of calcite is pertinent to crack repair and was scaled to 100 mL to yield sufficient product to characterize by scanning electron microscopy (SEM), energy dispersive spectroscopy (EDS), and X-ray diffraction (XRD). CA was prepared at 2.5 ug/mL enzyme in 0.1 M CAPS pH 11.0. CaCl₂ was added at a final concentration of 0.1 M to initiate calcite formation, and the pH was monitored continuously. Thermophilic SazCA-NBD (no binding domain) was used for the testing. Initially the reaction was very clear but after ˜6 hrs noticeable precipitation was observed, and the pH continuously dropped, indicating the enzyme reaction was proceeding. If the pH of the reaction dropped below pH 10, 1.0 M NaOH was added dropwise to bring the pH back to 11. The reaction was allowed to proceed for 48 hrs, which yielded a significant amount of precipitation in the beaker. These precipitates were vacuum filtered and dried, yielding ˜0.4 g of solid material.

The solid material was submitted for SEM, EDS and XRD analysis revealed crystals with a typical morphology of CaCO₃ crystals, and the EDS results demonstrate that the atomic composition is calcium, carbon, and oxygen, with a minor amount of iron and aluminum. XRD analysis revealed that the structure was mostly calcite, which is the crystal form of CaCO₃ associated with concrete strength. Data confirmed that the precipitates formed in the enzyme reaction are CaCO₃ and that they are produced in the calcite form.

### Example 3-Immobilization of Calcium Anhydride (CA) Enzyme on Silk Fibers

Experiments were conducted to investigate the immobilization of CA enzyme to biologically prepared fibers, which would allow the fibers to carry the crack repair functionality. For silk fibers, immobilization of CA was achieved by simple adsorption. Soluble SF was mixed at 4% weight with buffer at a 0.4% w/w ratio of enzyme to silk fiber. The presence of functional CA was determined by mixing the substrate for p-nitrophenyl acetate (pNPA) esterase activity with the silk in cuvettes and observing the color change, at a variety of concentrations. CA was first concentrated to avoid diluting the silk by adding more volume, which could affect β-sheet formation and subsequent stability of the silk.

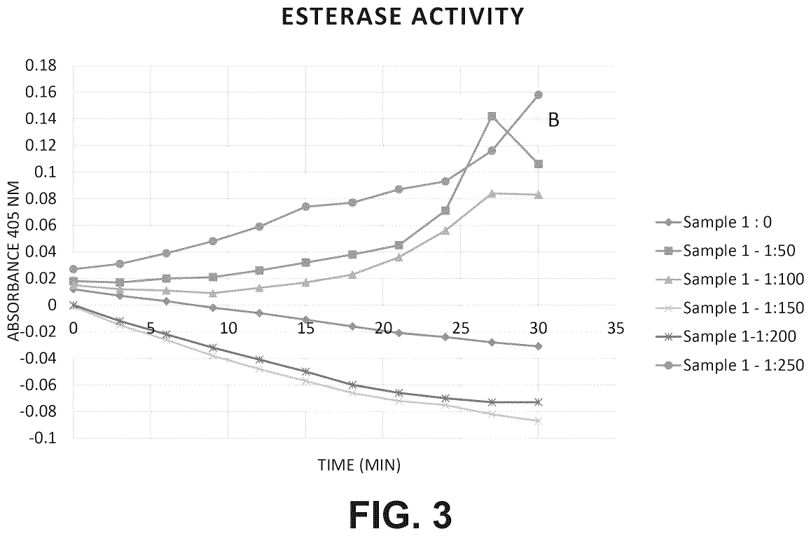

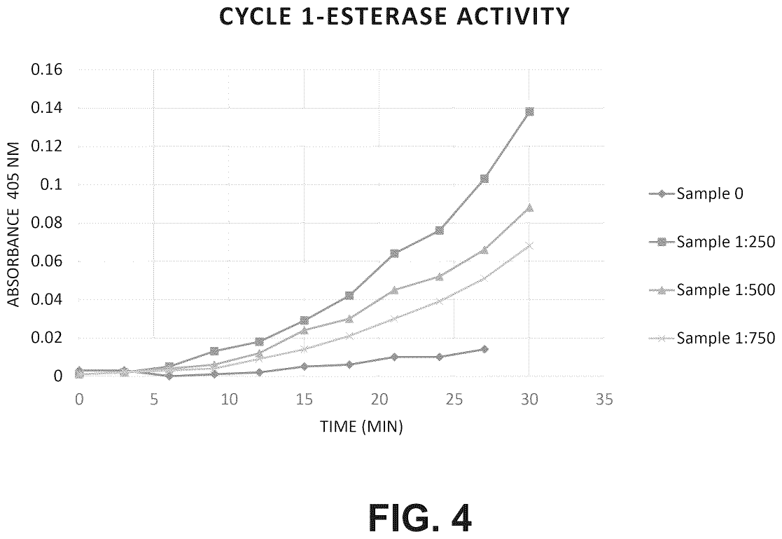

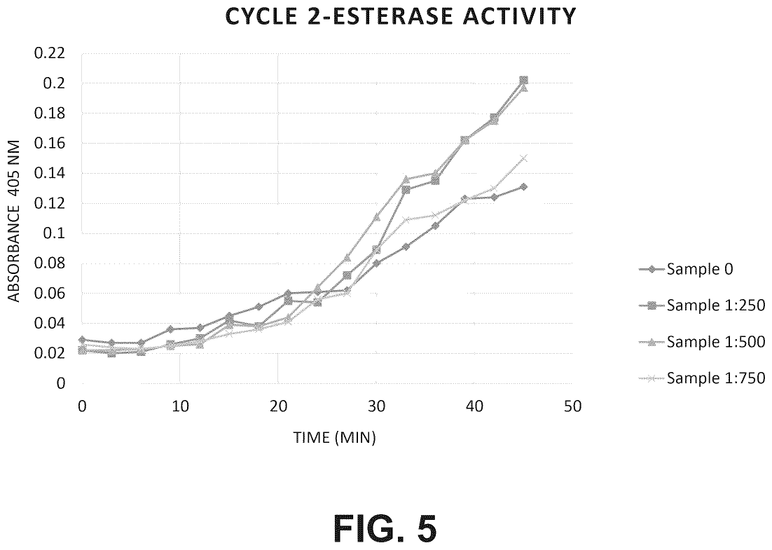

SF was used at 4 wt %, but with increasing concentration of CA enzyme, ranging from 1:50 wt/wt enzyme:silk at the highest CA concentration and 1:250 wt/wt enzyme:silk at the lowest CA concentration (FIGS. 3-5). Data confirms that 1:250 w/w SazCA-NBD:silk was the optimal formulation, the silk-enzyme fibers are functional, and the CA enzyme is stable for at least two cycles of esterase activity (Cycle 1 and cycle 2 data shown in FIGS. 4 and 5).

### Example 4-Immobilization of Carbonic Anhydrase (CA) Enzyme on Bacterial Cellulose (BC) Fibers

For Bacterial Cellulose (BC) fibers application, an assay was developed to estimate crack filling rates by quantitating CaCO₃ production over time. The 96-well plate format and the ability to monitor calcite production in real time makes this a convenient assay for evaluating various conditions (pH, calcium concentration, enzyme concentration, etc.) simultaneously, which allows us to quickly screen the different CAs for favorable crack repair properties.

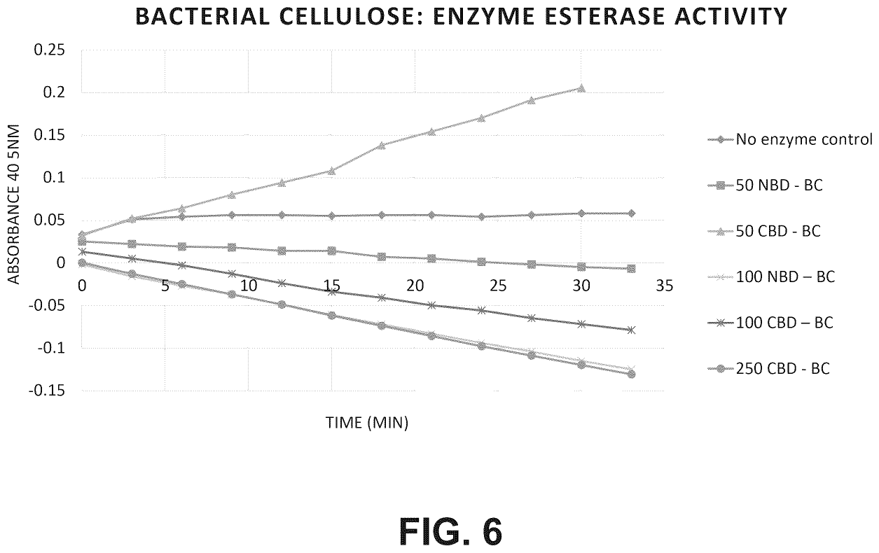

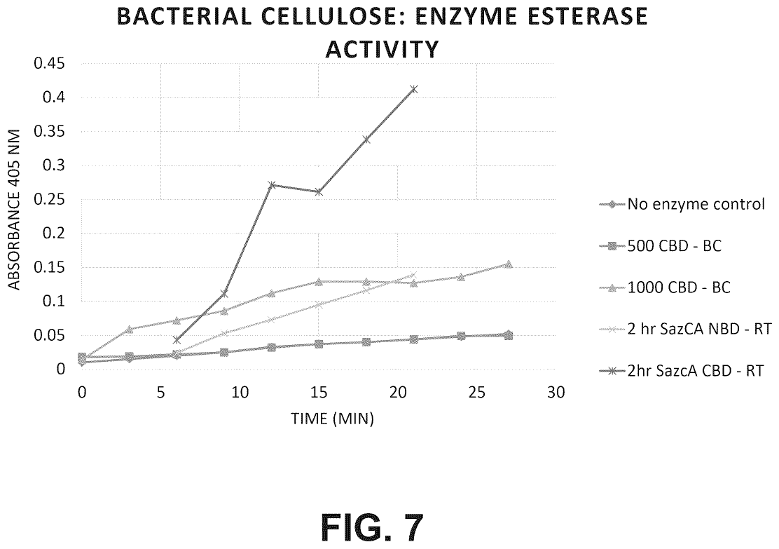

Samples were generated by taking soluble BC in NMMO and mixing with the appropriate ratio of enzyme and allowed to dry for 2 h. The presence of functional CA was determined by mixing pNPA substrate with the BC fibers and observing the color change (yellow color). BC was used at 0.7 wt % with increasing concentration of CA enzyme ranging from 1:50 wt/wt enzyme:BC at the highest CA concentration and 1:1000 wt/wt enzyme:BC at the lowest CA concentration. SazCA-NBD (no binding domain) and CBD (cellulose binding domain) were tested. Upon testing, the 1:50 wt/wt % ratio yielded the highest rate of activity for the pNPA assay with the series of dilutions after showing less activity (data related to BC examples are shown in FIGS. 6-7).

### Example 5-Evaluation of Calcite Formation in Concrete

Crack healing is driven by the catalytic reaction of CA with water, calcium ions (Ca²⁺) and CO₂ to produce calcite, which will self-assemble into a stable mineral that can incorporate with cementitious materials and fill cracks. The crack-healing rate is a measure of percent strength recovery in relation to time, evaluated as splitting tensile strength. After evaluation of crack-filling rate (time to fill the void), the biologically prepared fibers were tested for crack-healing rate (time to form calcite). Crack healing occurs when a supply of reagents is provided to the catalyst (i.e., providing calcium salts and CO₂ to CA). The reagents are applied by direct flow or injection to the top surface. Splitting tensile strength is then evaluated (triplicate microcosms) at various timepoints, and the failure mode observed visually as either cohesive within the SF or BC fibers, adhesive between fibers and concrete, or a mix of the fibers and concrete failure. The resulting percent recovery from splitting tensile strength measurements were plotted over time to determine the crack-healing rate.

CA was entrapped in silk fibers at a 1:250 wt/wt ratio and used to fill the 0.5 mm gap of fugitive microcosms. Based on observation and pNPA assay, the enzyme is active in the silk especially in enzyme:silk 1:250 ratio and on the concrete surface.

### Example 6-Crack-filling Rate Evaluation

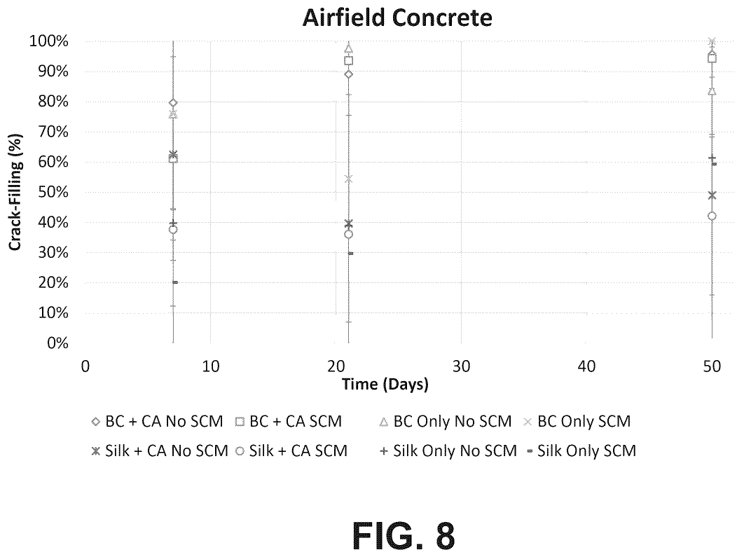

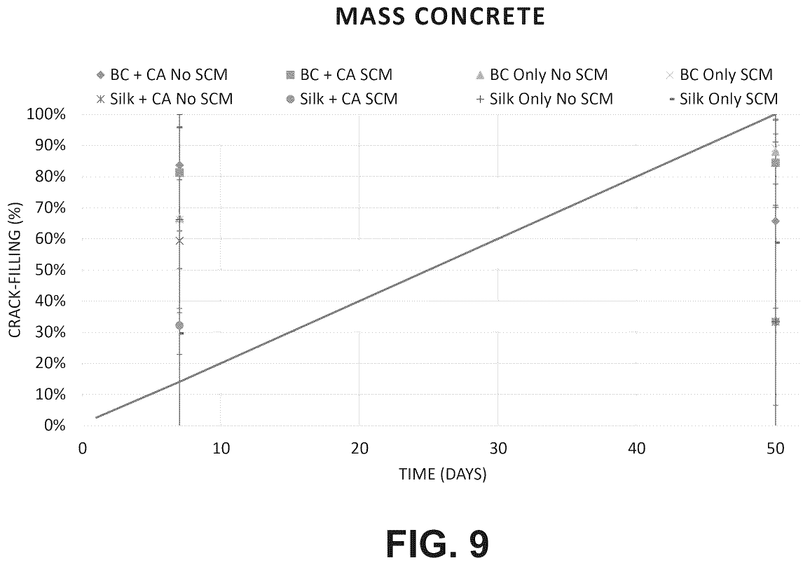

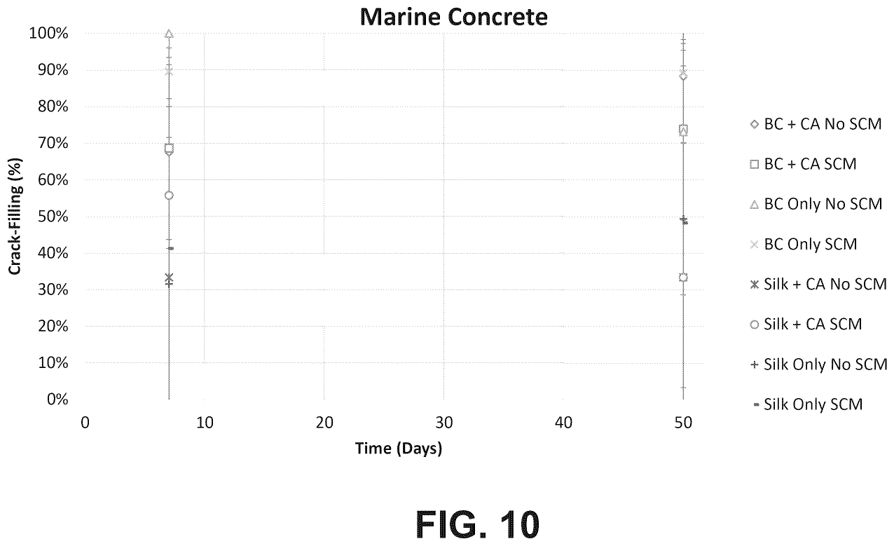

Crack-filling rate evaluation is tested on fugitive microcosms for marine concrete, mass concrete and airfield concrete (results are shown in FIGS. 8-10). Concrete samples containing supplementary cementitious material (SCM) and without SCM were tested. If the concrete type contains SCM, then supplementary cementitious material (SCM) was added to the original concrete formulation. The resulting data from measuring the axial cross section of the fugitive microcosms at various time points allow for calculation of the percentage of crack-filling per day with calcite. Crack-healing is determined on cracked microcosms using the splitting tensile strength test. The crack-healing rate is found by plotting the percent recovery in strength over time.

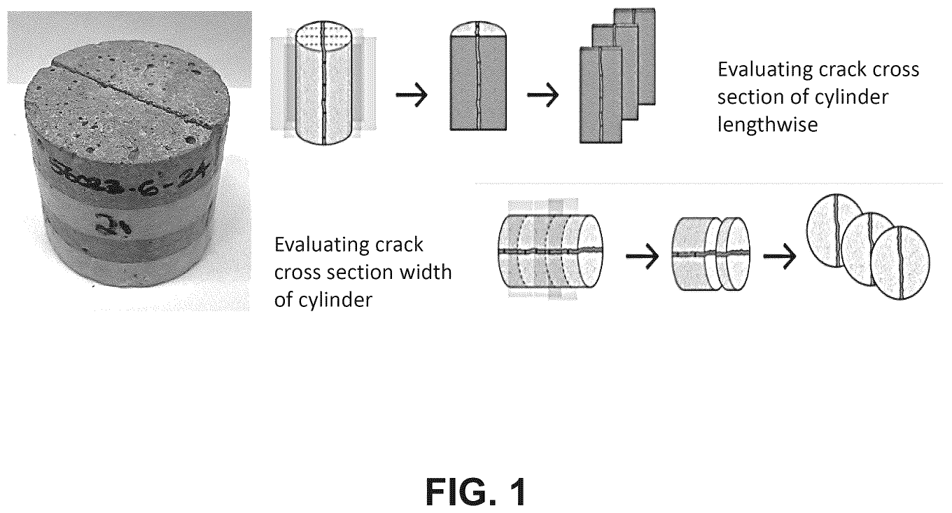

Fugitive microcosms contain a single manufactured crack to assess crack-filling rate. Two halves of a saw-cut cylinder were pieced together using spacers of a pre-determined thicknesses (0.2, 0.5 and 0.8-mm). Once reassembled, masking tape was applied to the circumference to ensure the specimen remains together and a plastidip coating was applied to all sides except for the formed or finished face. Fugitive microcosms were used to monitor crack-filling rate to evaluate metrics for applying function. The vascularization biologically prepared fibers were applied to the top surface (by injection for BC, by pipetting for SF) and allowed to fill the void. Crack repair function was then applied. At various timepoints the samples were sectioned with TechCut 4× Precision Low Speed Saw and analyzed for calcite formation. Off-center axial sections, and additional sections across the width of the cylinder were used for visualization of crack-filling rate and integration depth (See FIG. 1). Because integration along the depth is expected to primarily take place prior to biologically prepared fibers setting, sectioning across the width of the cylinder only occurred during the first timepoint (Day 1). Triplicate images were taken per concrete and treatment type. Prolonged stability and crack-filling via calcite formation at off center axial sections were evaluated at various timepoints over 50 days.

As seen in FIGS. 8-10, the vascularization material alone is capable of filling a portion of the cracked space. BC with CA fills greater than 60% of the crack width, and approaching 100% filled depending upon the type of concrete. Silk treatments, on the other hand, filled roughly 10-40% of the void space. This partial fill can be addressed with additional treatments.

With a minimum of a 13% fill of airfield concrete, the crack width was filled at a rate of at least 0.26 mL/(day-mLcrack) for airfield concrete in the first day. Mass and marine concrete achieved crack-filling rates of greater than or equal to 0.23 and 0.09 mL/(day-mLcrack), respectively.

Those skilled in the art will understand from the foregoing description that modifications and changes may be made in various embodiments of the present disclosure without departing from its true spirit of the invention. The descriptions in this specification are for purposes of illustration only and are not to be construed in a limiting sense.

Various other embodiments are disclosed and detailed in the following experimental examples, figures, description, data/tables presented below.

What is claimed is:

1. A formulation for repair of a structure, the formulation comprising:
biologically prepared fibers;
a solvent; and
optionally at least one enzyme;
wherein the biologically prepared fibers form a three-dimensional fibrous scaffold configuration within the structure.

2. The formulation of claim 1, wherein the structure comprises cementitious material, natural rock, mortar, concrete, or a combination thereof

3. The formulation of claim 1, wherein the biologically prepared fibers are selected from silk fibroin, bacterial cellulose, or a combination thereof.

4. The formulation of claim 1, wherein the biologically prepared fibers comprise silk fibroin and the biologically prepared fibers are present in the formulation at 4-15% wt/vol %.

5. The formulation of claim 1, wherein the biologically prepared fibers comprise silk fibroin and the biologically prepared fibers are present in the formulation at about 8-10% wt/vol.

6. The formulation of claim 1, wherein the biologically prepared fibers comprise water-insoluble silk II proteins.

7. The formulation of claim 6, wherein the silk II proteins are present in β-sheet formation.

8. The formulation of claim 1, wherein the biological fibers comprise bacterial cellulose and the solvent comprises N-Methylmorpholine-N-Oxide (NMMO).

9. The formulation of claim 1, wherein the at least one enzyme catalyzes mineral formation within the void of the structure.

10. The formulation of claim 1, wherein the at least one enzyme comprises carbonic anhydrase.

11. The formulation of claim 3, wherein bacterial cellulose fibers are present in the formulation at 0.4-1.5% wt/vol %.

12. A method of repairing a structure, the method comprising:
applying a first solution comprising biologically prepared fibers to the structure;
optionally, applying a second solution containing at least one enzyme to the structure;
wherein the biologically prepared fibers comprise Silk Fibroin (SF), or Bacterial Cellulose (BC), or a combination thereof.

13. The method of claim 12, wherein the structure comprises cementitious material, natural rock, mortar, concrete, or a combination thereof

14. The method of claim 12, wherein the biologically prepared fibers comprise silk fibroin and the biologically prepared fibers are present in the first solution at about 4-15% wt/vol.

15. The method of claim 12, wherein the biologically prepared fibers comprise water-insoluble Silk II proteins.

16. The method of claim 15, wherein the silk II proteins are present in β-sheet formation.

17. The method of claim 12, wherein the first solution comprising comprises bacterial cellulose and a solvent comprising N-Methylmorpholine-N-Oxide (NMMO).

18. The method of claim 12, wherein the second solution comprises least one enzyme that catalyzes mineral formation within the void of the structure.

19. The method of claim 12, wherein the at least one enzyme comprises carbonic anhydrase.

20. A method of repairing a structure, the method comprising:
preparing a solution comprising:
biologically prepared fibers;
a solvent; and
at least one enzyme; and
applying the solution to the structure in a single application step;
wherein the biologically prepared fibers comprise Silk Fibroin (SF), or Bacterial Cellulose (BC), or a combination thereof.
